(19) United States
(12) Patent Application Publication
(10) Pub. No.: US 2026/0009875 A1
(43) Pub. Date: Jan. 8, 2026

(54) METHOD FOR POST-PROCESSING A SEQUENCE OF ACQUISITION OF PERFUSION BY A MEDICAL IMAGING DEVICE

(71) Applicant: OLEA MEDICAL, La Ciotat (FR)

(72) Inventor: Habib REBBAH, LYON (FR)

(73) Assignee: OLEA MEDICAL, La Ciotat (FR)

(21) Appl. No.: 18/993,426

(22) PCT Filed: Jul. 6, 2023
(86) PCT No.: PCT/FR2023/051043

§ 371 Date: Jan. 10, 2025

(30) Foreign Application Priority Data
Jul. 13, 2022 (FR) FR2207242

(51) Int. Cl. G01R 33/563; A61B 5/00; A61B 5/026; G06N 3/0464; G06N 20/00; G06T 7/00

(52) U.S. Cl. CPC G01R 33/56366; A61B 5/0263; A61B 5/7267; G06N 3/0464; G06N 20/00; G06T 7/0012; G06T 2207/30104

(57) ABSTRACT

A method for post-processing a sampled time-dependent experimental perfusion signal to generate a pharmacokinetic parameter is implemented by a processing unit of a medical-imaging analysis system, said unit having been trained beforehand in a process allowing the disrupting effect of acquisition of a perfusion sequence on arterial signals and redundancy of information related to an arterial input function shared by a set of at least two tissual signals to be learnt. Such an arterial input function is produced directly in a step by said processing unit thus trained from a first arterial input function and from tissual signals selected beforehand.

The invention relates to a method for post-processing a perfusion acquisition sequence by a medical imaging device. The latter can be a magnetic resonance imaging, or MRI, device, or an imaging device based on the use of X-rays, such as a CT scanner. Such a post-processing method makes it possible to estimate a sampling of the arterial input function, also known by the acronym AIF. This sampling has the distinctive characteristic of being much less sensitive to the acquisition effects and thus of preserving the linearity with regard to the concentration of the contrast product, which is essential for a quantitative estimation of the pharmacokinetic parameters, which makes it possible, ultimately, to characterize lesions such as tumours or ischaemic tissues.

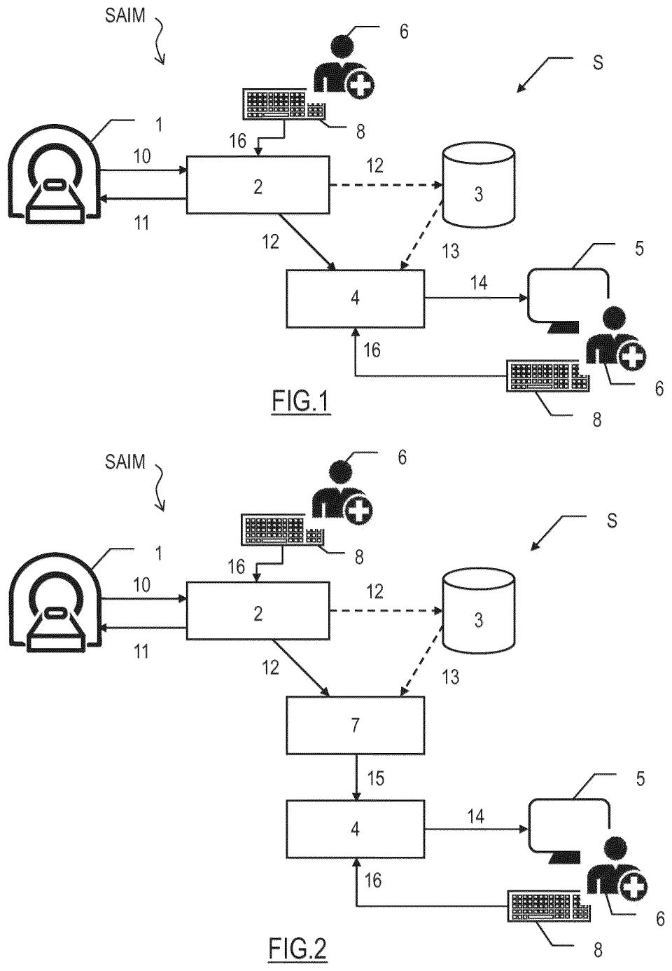

Magnetic resonance imaging is based on an analysis of the response of the proton of a water molecule when it is excited in a magnetic field. This response depends on the environment of such a proton and thus makes it possible to differentiate different types of tissues. A nuclear magnetic resonance imaging device, such as the device 1 of a medical imaging analysis system SAIM illustrated by way of non-limitative example in FIGS. 1 and 2, is generally used. This delivers a plurality of sequences of digital images 12 of one or more parts of the body of a patient, by way of non-limitative examples, the brain, the heart, the lungs. To this end, said device applies a combination of high-frequency electromagnetic waves to the part of the body in question and measures the signal re-emitted by certain atoms, such as by way of non-limitative example, hydrogen for nuclear magnetic resonance imaging. The device thus makes it possible to determine the magnetic properties and, consequently, the chemical composition of the biological tissues and therefore their nature, in each elementary volume, commonly called a voxel, of the imaged volume. As shown in FIG. 1, a nuclear magnetic resonance imaging device 1 is controlled by means of a console 2. A user 6, for example an operator, practitioner or researcher, can thus choose commands 11 to control the device 1, on the basis of parameters or instructions 16 input via an input human-machine interface 8 of the analysis system. Such a human-machine interface 8 can consist for example of a computer keyboard, a pointing device, a touchscreen, a microphone or, more generally, any interface arranged to express a gesture command or an instruction issued by a human 6 as control or parameterizing data. On the basis of items of information 10 generated by said device 1, a plurality of digital image sequences 12 are obtained of a part of a body of a human being or an animal. Such items of information 10 or images 12 will also be called “experimental data”.

A CT scanner in contemporary radiology shares several similarities of use with a magnetic resonance imaging device as illustrated in FIGS. 1 and 2. These latter could describe a variant of a medical imaging analysis system SAIM incorporating an imaging device 1 in the form of a CT scanner in place of a magnetic resonance imaging device. The invention will be described below principally with the aim of resolving an acquisition effect by means of a magnetic resonance imaging device. However, a specific description can apply to other imaging devices (such as the CT scanner) when necessary.

The image sequences 12 can optionally be stored within a server 3, i.e. a computer equipped with its own storage means, and constitute a medical file 13 of a patient. Such a file 13 can comprise images of different types, such as functional images demonstrating the activity of the tissues, or anatomical images reflecting the properties of the tissues. The image sequences 12 or, more generally, the experimental data are analyzed by a processing unit 4 arranged for this purpose. Such a processing unit 4 can, for example, consist of one or more microprocessors or microcontrollers implementing appropriate application program instructions loaded into storage means of said imaging analysis system. By “storage means” is meant any volatile or, advantageously, non-volatile computer memory. A non-volatile memory is a computer memory the technology of which makes it possible to retain its data in the absence of an electrical energy supply. It can contain data resulting from inputs, calculations, measurements and/or program instructions. The main non-volatile memories currently available are of the type capable of being written to electrically, such as EPROM (erasable programmable read-only memory), or also written to and erased electrically, such as EEPROM (electrically erasable programmable read-only memory), flash, SSD (solid-state drive), etc. Non-volatile memories are distinguished from the memories known as “volatile”, from which the data are lost in the absence of an electrical power supply. The main volatile memories currently available are of the RAM (random access memory, also called “read-write memory”), DRAM (dynamic random-access memory, needing to be regularly refreshed), SRAM (static random-access memory needing to be refreshed in this way when there is a loss of electrical power), DPRAM or VRAM (particularly suitable for video), etc., type. In the rest of the document, a “data memory” can be volatile or non-volatile according to the target application.

Said processing unit 4 includes means for communicating with the outside world for collecting the images. Moreover, said communication means make it possible for the processing unit 4 ultimately to deliver or output, a rendering, for example graphical and/or acoustic, of an estimation or a quantification of a biomarker or of a pharmacokinetic parameter QI created by said processing unit 4 on the basis of the experimental data 10 and/or 12 obtained by magnetic resonance imaging, to a user 6 of the imaging analysis system by means of an output human-machine interface 5. Throughout the document, by “output human-machine interface” is meant any device, used alone or in combination, making it possible to output or to deliver a graphical, haptic, acoustic representation or, more generally, one that can be perceived by a human being, of a reconstructed physiological signal, in this case a biomarker, to a user 6 of a magnetic resonance imaging analysis system. Such an output human-machine interface 5 can consist non-exhaustively of one or more screens, speakers or other suitable alternative means. Said user 6 of the analysis system can thus confirm or invalidate a diagnosis, decide on a therapeutic action that they consider appropriate, undertake further research, refine adjustment parameters of an item of measuring equipment, etc. Optionally, this user 6 can also parameterize the operation of the processing unit 4 or of the output human-machine interface 5, by means of operating and/or acquisition parameters 16. For example, they can thus define display thresholds or choose the biomarkers, indicators or estimated or quantified parameters of which it is desired to have a representation. To this end, the user utilizes the input human-machine interface 8 mentioned above or a second input interface provided for this purpose. Advantageously, the input 8 and output 5 human-machine interfaces can constitute one and the same physical entity only. Said input 8 and output 5 human-machine interfaces of the imaging analysis system can also be integrated into the acquisition console 2. A variant exists, described in relation to FIG. 2, for which an imaging system, as described above, also includes a pre-processing unit 7 for analyzing the image sequences 12, deducing experimental signals 15 therefrom, and delivering these latter to the processing unit 4 which is thus relieved of this task.

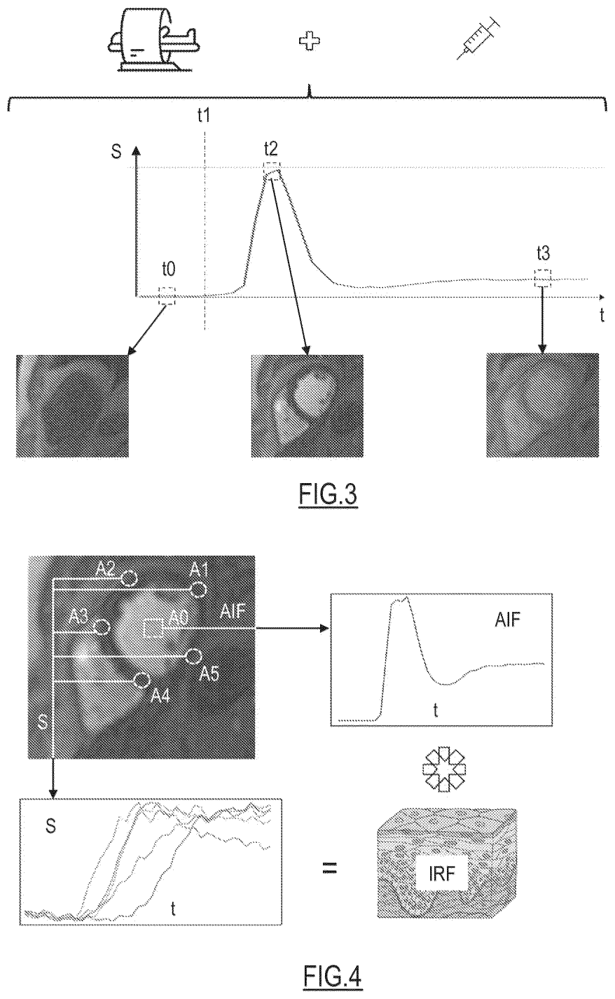

Among the techniques or methods based on magnetic resonance imaging, perfusion magnetic resonance imaging can be distinguished. Such a technique, shown diagrammatically in FIG. 3 which illustrates a cardiac perfusion acquisition sequence, consists of applying a radiofrequency pulse called a magnetization preparation radiofrequency pulse to allow the latter to move towards its steady state (it can also make it possible to do away with the magnetization history), followed by a series of acquisition radiofrequency pulses to spatially sample the volume of interest. The set of parameters chosen by the technician has the aim of maximizing or minimizing different effects, among the most significant can be found the choice of the type of pulses making it possible either to obtain a sequence called T1-weighted enhancement or T2*-weighted susceptibility. During the acquisition, the technician carries out the injection of a tracer in the form of a contrast product into the venous system of the patient undergoing the examination. The magnetization of the volume of interest thus varies during the passage of the latter-distributed throughout the body by means of the circulatory system—and this variation depends on the quantity of the tracer. FIG. 3 thus illustrates at three separate moments t0, t2, t3, such a change in the magnetization of the volume of interest, respectively upstream and downstream of the arrival at the moment t1 of the tracer in the volume of interest. The perfusion sequence thus consists of regularly acquiring the magnetization state of the volume of interest by repeating quasi-periodically the application of an acquisition radiofrequency pulse which can be preceded in certain cases by a preparation pulse. A perfusion sequence thus makes it possible to obtain, for each elementary volume or voxel, a temporal sampling S(t) of the variation in the magnetization state resulting from the acquisition parameters selected by the technician.

The case of the CT scanner differs from the MRI by its basic principle: the greater or lesser opacity of the anatomical structures passed through by X-rays. The principle of the perfusion sequence thus consists of injecting a contrast product making it possible to modify this opacity. The main disruptive effect that may be mentioned being a subsampling of the AIF.

As described in FIG. 4, the estimation of the pharmacokinetic parameters is based on the theory of tracer dilution which makes it possible to link the AIF to the concentration of the contrast product S (relative to five regions of interest A1 to A5 as shown in FIG. 4) in the tissue by means of a convolution product with a filter called “impulse response” or “IRF” for “impulse response function” in relation to the recirculation of said contrast product, such that S=AIF*IRF. FIG. 5 thus illustrates five temporal signals S respectively associated with said regions A1 to A5.

By retrieving a sampling of the concentration of the contrast product in the tissue directly in the acquired images and choosing a relevant sampling of the AIF, it is possible to estimate the IRF and thus the pharmacokinetic parameters.

The perfusion sequences make it possible to expose lesions which are invisible in the native state, which proves to be very useful for a practitioner seeking to establish a diagnosis and to make a therapeutic decision in the treatment of pathologies. However, in order to perfect the decision-making, whether in the clinic or in the field of research, it is necessary to obtain an AIF subjected to few acquisition effects. FIG. 5 describes a signal S acquired by a perfusion sequence depending on the concentration C of the contrast product or tracer according to a plurality of acquisition parameters, which explains the plurality of curves S. In fact, magnetic resonance imaging being a capture of a magnetization state of the tissues, the presence of a contrast product acts indirectly on the acquired signal. The contrast product will influence the speed of return to the steady state of the magnetization. In other words, for one and the same set of acquisition parameters, the magnetization will vary depending on the concentration of the product injected into the patient. This relationship is neither linear nor even bijective. However, as indicated by the right-hand view described in FIG. 5, in the case of a low dose of contrast product, it is usual to consider the relationship between the acquired signal S and the concentration C of the product as linear. Such a right-hand view illustrates an enlargement for low concentrations C of contrast agent. Regardless of the acquisition parameters, the curves S and C merge with the straight line identified as DI represented by a dashed line within a zone ZL called linearity zone in grey in FIG. 5. For low concentrations

C, it is thus possible to use the signals S acquired directly without correcting the acquisition effects, which is generally accepted for the tissue signals. However, as indicated by the curves in the left-hand view in FIG. 5, the higher the concentration C, the greater the acquisition effect. The approximation accepted for the tissue signals may therefore not apply for the arterial signals characterized by a maximum concentration of the contrast product. It is therefore necessary to correct the acquisition effect in order to estimate relevant AIF samplings.

At this point in time several post-processing methods have been proposed in the literature for correcting this acquisition effect, like that disclosed in the article “Physics-informed neural networks for myocardial perfusion MRI quantification”, van Herten et al. 2022. Some of them are little used such as those relating to the use of an AIF called population AIF (using an AIF database corresponding to a given injection protocol and to a type of patient) or those requiring a blood sample simultaneous to the acquisition in order to really know the quantity of contrast agent present at a given moment. On the other hand, two methods that the invention directly competes with, as well as a third which the invention proposes to improve, are to be examined.

A first known method consists of digitally simulating the acquisition effects by means of physical models derived from Bloch equations. These models require a very detailed knowledge of the perfusion sequence and are therefore to be reevaluated for each case. In addition, the acquired signals S for one and the same perfusion examination will depend on parameters intrinsic to the tissues which generated them, in particular the T1 and the T2* which will therefore need to be estimated by means of other acquisition sequences and evaluated by post-processing methods necessarily having a bias. The acquisition of additional parameters, the value of which varies for each voxel of the image, thus introduces a source of error and a new problem to be solved: the multimodal registration between the perfusion images and the T1 and T2 maps. Finally, the set of parameters necessary for this method is not necessarily stored in routine clinical practice and is thus often lost, limiting future reuse of the perfusion acquisitions.

A second method proposes to use a set of acquisition parameters making it possible to greatly limit the acquisition effects and thus to consider the arterial signals as viable AlFs. However, such an approach is to the detriment of the image resolution. Consequently, it is impossible to use such images to very accurately estimate pharmacokinetic parameters. In fact, this acquisition is combined with a conventional perfusion acquisition proposing a resolution that is sufficient for the evaluation of these parameters by using the AIF obtained means of the arterial signals of the acquisition optimized to this end. This method has the advantage of allowing the direct use of the signals originating from the different acquisitions. However, it assumes a technician trained in this type of parameterization and the availability of this type of manipulation on the MRI machine, which is currently still uncommon. In addition, this method assumes that the two types of acquisitions are obtained simultaneously. In other words, this method cannot involve any historic perfusion acquisition. However, over more than thirty years, numerous databases using conventional acquisitions have been formed. This method ends up excluding them.

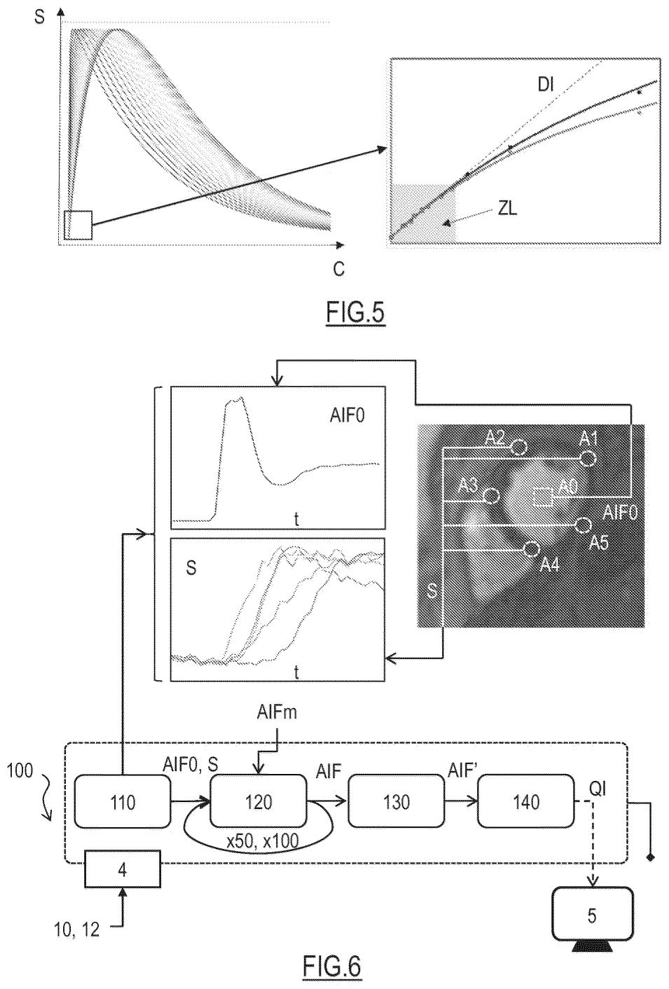

Finally, a third method, illustrated in FIG. 6, proposes to use all the redundant information shared by tissue signals to estimate an AIF sampling. The signal of each voxel describing regions of interest of the tissue, such as the regions A1 to A5 illustrated by dashed circles by way of example in FIG. 6, being the product of convolution between the IRF belonging to each voxel and the AIF shared by all the tissue voxels, it is possible to simultaneously estimate the IRF of each voxel and the AIF of all the voxels. This approach is denoted by the term blind deconvolution, since neither the IRF nor the AIF are known. Such a method 100, for example implemented by a processing unit 4 of a medical analysis system illustrated in FIG. 1 or 2 supplied with experimental data 10 and/or 12, firstly consists of a step 110 of selecting tissue signals S and of a first arterial input function AIF0 generally in an arterial region A0 symbolized in FIG. 6 by a dashed square, then of a step 120 of iterative minimization of the residual errors between measured tissue signals S and those reconstructed on the basis of a parametric model AIFm of arterial input function AIF. The objective is to adjust the parameters of said arterial input function model AIFm in order to minimize said residual errors. A very wide variety of methods for performing this minimization 120 have been described in the literature. They are generally differentiated by the constraints that they impose on said arterial input function model. Certain methods are said to be “without constraints” because the sampling of the arterial input function can take any form. This first type of method is little used because it is very sensitive to acquisition noise. Other methods are parameterized by a given model. However, this second type of method is limited by the nature of the model of the arterial input function used, thus restricting the forms that an arterial input function can take.

The choice and the number of tissue signals selected in step 110 are variable according to the method selected. If it was shown that, theoretically, their number could be reduced to two, including for methods not requiring any constraint on the form of the arterial input function sought, limiting this number of signals to two assumes a very large number of hypotheses that are not very realistic in practice, in particular relating to the effect of the noise and the type of perfusion model describing these two signals. As a general rule, said number of tissue signals used in the literature varies from six to twelve.

Said second step 120 of minimizing errors commonly consists of minimizing the quadratic sum of the residual errors between the signals chosen in step 110 and those reconstituted on the basis of the adjustment of the parameters of the arterial input function model and of the pharmacokinetic parameters. It is an iterative step 120 which thus consists of searching for this set of parameters which makes it possible to reduce this total error. The adjustment is habitually made by a gradient descent method and each iteration involves a large number of mathematical operations that are costly in terms of calculating time. The stop criterion that thus determines the final number of iterations is variable from one study to the other but can relate to a minimal change in the error from one iteration to another or even directly to the value of the error. In the literature, methods can be found proposing from fifty to several hundreds of iterations causing an implementation time of several minutes. Once this second step has been carried out, the method makes it possible to generate a second arterial input function AIF which will be qualified as “viable” for the set of tissue signals chosen. However, by the very nature of the method, the latter only represents the correct form of the second arterial input function AIF sought. Thus it is necessary to implement a step 130 consisting of adjusting the scale of the AIF (height of the sampling). Multiple ways of carrying out this step of scaling or normalization exist in the literature. For example, it can be assumed that the AIF sought shares the area under the curve of AIF0 or, at least, a part that has been subjected to few acquisition effects. The second corrected arterial input function AIF′ thus obtained can now be used over all or a portion of the voxels of the perfusion image to estimate in a subsequent step 140 one or more pharmacokinetic parameters QI thereof and thus create information relevant for making therapeutic decisions. Such pharmacokinetic parameters QI can be the subject of the optional formation of one or more graphic representations and be the subject of an output via an appropriate human-machine interface, such as the interface 5 in relation to the medical imaging analysis system according to FIG. 1 or 2, so a member of healthcare personnel can perform a diagnosis for example.

The invention makes it possible to overcome all or part of the drawbacks raised by the known or aforementioned solutions.

Among the numerous advantages provided by the invention, there may be mentioned the implementation of a post-processing method which allows:
a free form, i.e. unconstrained, for the arterial input function that is not very sensitive to the acquisition noise;
a reduction in the number of tissue signals required for the implementation of the method;
a non-iterative execution of the estimation of the arterial input function making it possible to obtain execution times of the order of the second on a standard calculating machine for the general public versus several minutes according to the state of the art.

To this end, the invention relates to a method for post-processing a sampled temporal experimental signal resulting from a perfusion acquisition sequence by a medical imaging device and resulting from the passage of a tracer within an elementary volume of an organ, said method being implemented by a processing unit of a medical imaging analysis system. Such a method includes:
a step of selecting a first arterial input function in relation to an arterial region of the organ and a set of tissue signals respectively in relation to separate tissue regions of said organ;
a step of generating a second arterial input function on the basis of said first arterial input function and of said set of selected tissue signals;
a step of creating a pharmacokinetic parameter on the basis of said second arterial input function and of said experimental signal.

In order to augment the implementation performance of such a method with regard to known techniques, the step of generating a second arterial input function according to the invention consists of the implementation of basic operations by said processing unit trained beforehand according to a process of learning the disruptive effect of the acquisition of a perfusion sequence on arterial signals and the redundancy of the information in relation to such a second arterial input function shared by a set of at least two tissue signals.

According to a particular embodiment, such a method can include a step of correction by scaling said second arterial input function, generated before the implementation of the step of creating a pharmacokinetic parameter on the basis of said second arterial input function thus corrected and of said experimental signal.

Advantageously, and in order to offer a member of healthcare personnel the possibility of verifying the relevance of said second arterial input function generated when said medical imaging system comprises an output human-machine interface, a method according to the invention can include a step of creating a graphic representation of said second arterial input function and of outputting said graphic representation by means of said output human-machine interface.

According to a preferred embodiment, the learning process can consist of deep learning based on minimization of the average value of the quadratic errors between real samples of arterial input functions which have made it possible to generate tissue signals and an estimation of these same samples performed by said learning process, such a learning process being capable of being carried out via the Adam optimizer.

According to a second subject, the invention relates to a computer program product including one or more program instructions that can be executed by the processing unit of a computer, said program instructions being capable of being loaded into a non-volatile memory of said computer and the execution of which by said processing unit causes the implementation of a post-processing method according to the invention.

Similarly, according to a third subject, the invention relates to a computer-readable storage medium including the instructions of such a computer program product.

Finally, the invention moreover relates to a medical imaging analysis system including a processing unit arranged to communicate with the outside world and receive a set of samples of a temporal experimental signal resulting from a perfusion acquisition sequence by a medical imaging device and resulting from the passage of a tracer within an elementary volume of an organ. Such a processing unit has been trained beforehand according to a learning process previously mentioned and includes storage means comprising the program instructions of a computer program product according to the invention.

Other characteristics and advantages will become more clearly apparent on reading the following description and examination of the accompanying figures in which:

FIG. 1, already described, illustrates a medical imaging system comprising a medical imaging platform;

FIG. 2, already described, illustrates a variant of such a medical imaging system comprising a medical imaging platform;

FIG. 3, already described, describes a sequence of cardiac perfusion by medical imaging;

FIG. 4, already described, illustrates an example of a known process of estimating impulse responses in relation to voxels of interest and thus of generating pharmacokinetic parameters;

FIG. 5, already described, illustrates the disruptive effect of an acquisition on the linearity of the signal according to the concentration of the contrast product;

FIG. 6, already described, illustrates a known method for obtaining an estimation of arterial input function by blind deconvolution;

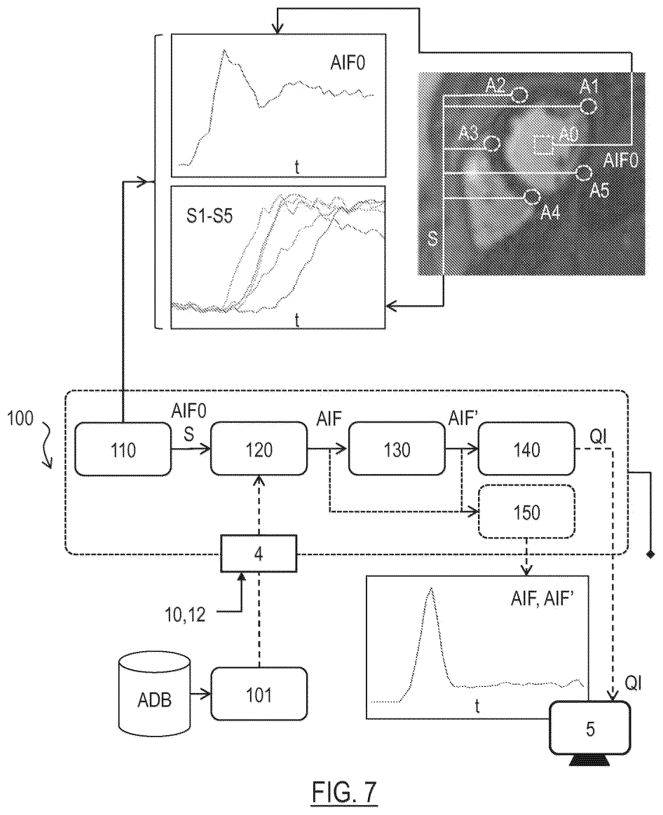

FIG. 7 illustrates a method for generating a viable arterial input function by blind deconvolution and ultimately pharmacokinetic parameters of interest according to the invention.

As shown in FIG. 7, the invention relates to a method 100 for generating a pharmacokinetic parameter of interest (QI) on the basis of one or more sampled temporal experimental signals (10, 12) originating from an acquisition by a perfusion medical imaging device (1). Such a method (100) can be implemented by the processing unit (4) of such a medical image analysis system. Such a method (100) relies on a process of learning (101) the disruptive effect of the acquisition of the perfusion sequence on the arterial signals combined with learning the redundancy of the information in relation to said arterial input function shared by a set of at least two tissue signals. The processing unit 4 is thus arranged to include a convolution neural network or any other equivalent solution, having been configured beforehand by the implementation of said learning process 101. In fact, said tissue signals all originate from the convolution product of one and the same arterial input function AIF and from their respective impulse responses IRF. It is thus possible to learn to extract therefrom the shared information which is actually the arterial input function AIF sought. The learning of the disruptive effect of the acquisition is itself carried out by supplying an arterial signal AIF0 which is actually a degraded version of the AIF sought. Thus, it will be a question of learning to “transform” such a selected arterial signal AIF0 such that the latter can correspond to the information shared by the tissue signals, avoiding proposing solutions which are very unlikely. In other words, this point promotes the stability of the algorithm. This learning process 101 can be carried out using a combination of signals acquired physically from volunteers, patients or using physical phantoms and/or simulated signals making it possible to generate a wider variety of cases and thus promoting the ability to extrapolate for the method of generation of a viable arterial input function AIF sought as such. By using a database ADB thus generated, the learning 101 can for example be carried out using a deep learning algorithm. The estimation of the learning algorithm parameters can be guided by a minimization of the estimation error of the AIF sought and that having been used to generate the tissue signals of the database ADB. Said estimation can also be directly guided by minimizing the error between said tissue signals originating from the database ADB and the tissue signals estimated by the algorithm if a step of reconstructing said signals by means of the AIF′ generated by the algorithm is proposed. Thus, the database ADB can be optionally constituted by:
the AIFs sought, their transforms AIF0 subjected to the acquisition effects and the signals that they generate;
the AIF0s subjected to the acquisition effects and the signals generated by the AlFs sought.

Once the step 101 of learning makes it possible to achieve satisfactory performance, the post-processing method 100 can make use of a convolution neural network (or any other equivalent solution), called “network” below, thus trained within the processing unit 4 implementing said method 100. The latter includes, like the post-processing method 100 described in relation to the third method according to the state of the art, a step 110 of selecting an initial proposal of arterial input function AIF0 and a set of tissue signals S that do not correspond to blood vessels or to a void. Such a step 110 can be carried out according to different techniques. Such a selection can thus consist of choosing an average arterial input function originating from a known population. It can also result from a manual selection by the technician on the image resulting from the acquisition of a sampled temporal experimental signal of several zones of samplings from which this arterial input function results, as well as the set of tissue signals, or from the implementation of any post-processing method making it possible to automatically deduce this arterial input function and the set of tissue signals from the image. Said method 100 includes a step 120 of generating an estimation of a second viable arterial input function AIF for the set of input signals not subjected to the acquisition effects of the initial proposal of arterial input function AIF0. Unlike said previously known method 100, the step 120 of a method 100 according to the invention consists of directly generating, i.e. without implementing a costly iterative process 120, a second viable arterial input function AIF on the basis of basic operations implemented by a network trained according to the process 101. The step 120 is thus drastically quicker and simpler to implement than the iterative solution according to the state of the art. A method 100 according to the invention can also include a step 130 of scaling said second arterial input function AIF generated so that it is ultimately used after correction in a step 140 of generating one or more pharmacokinetic parameters of interest QI for all or a portion of the voxels of a perfusion image, these latter being able to be the subject of an output, for example in a graphic form via an output human-machine interface, such as the interface 5 of a medical imaging analysis system SAIM according to FIG. 1 or 2. However, the invention provides that the step 130 of normalization of the AIF generated can also be integrated directly into the step 120 directly generating an AIF′ that is viable and normalized, i.e. scaled and available to implement the step 140 of generating one or more pharmacokinetic parameters of interest QI for all or a portion of the voxels of a perfusion image. A relevant scaling factor could be calculated in such a way as to respect a preservation of the mass between the arterial input function generated and said experimental signal, for example by making a low distortion zone correspond in the two signals.

An implementation example of the learning process 101 of a network according to the invention which has made it possible to validate the relevance of the invention is to be examined.

As mentioned above, a database ADB can be constituted by more than three million AIF samples simulated by means of a random variation of the parameters of the model called Parker's model. Each AIF sample can thus be used to generate five tissue signals with different pharmacokinetic parameters. The model used to generate these signals can be the model called the Toft-Ketty model augmented by the addition of a delay between the sampling of the AIF and that of the tissue.

A deep learning algorithm is selected to generate a second viable arterial input function AIF in a step 120 of a post-processing method 100 according to the invention. Such a deep learning algorithm is advantageously divided into two branches. The first branch processes the sampling of the arterial input function AIF0. It is made up of three layers of one-dimensional convolution networks. The second branch processes the set of tissue signal samplings. It is made up of three layers of two-dimensional convolution networks. The two branches each end with a network of simple neurons having a single layer then grouped together by a network of simple neurons having three layers. The last layer proposing at output a sampling of arterial input function AIF of the same size as that of the initial proposal of arterial input function AIF0.

Advantageously, the learning process 101 can be carried out by an optimizer called Adam optimizer based on a minimization of the average value of the quadratic errors between the real samples of arterial input functions which have made it possible to generate the tissue signals and the estimation of these same samples performed by the learning algorithm. The number of iterations used for this training was thirty with reference to eighty percent of the database generated, the validation thus being carried out on the remaining twenty percent. The results obtained from validation, in terms of mean squared error, were 0.69% of the value of the peak of the true AIF samples for the training database as well as for that used for the validation. This approach is generally described in the literature as having the best results. However, this deep learning can be replaced by a table creation method allowing correspondence to be made between an input, represented by an initial arterial input function and a set of tissue signals, and an output representing a second arterial input function not subjected to the acquisition effects. In this case, the learning consists of setting up such a table.

So as to guarantee a clinical viability of a post-processing method 100 according to the invention, this same deep learning algorithm has been tested on an MRI cardiac perfusion imaging database constituted by forty-three elements. These images have the distinctive characteristic of having been acquired using the second method of the state of the art described above. In doing so, the available arterial input function samples were subjected to few acquisition effects associated with each conventional acquisition. By manually selecting the region of the myocardium to extract therefrom five samplings of tissue signals and the region of the left ventricle to extract therefrom a proposal of arterial input function sampling AIF0 subjected to many acquisition effects, the learning algorithm was able to be evaluated on real data by comparing the estimations of arterial input function samplings carried out by the latter with the samplings subjected to few acquisition effects. The results have been measured in terms of mean squared error and of coefficient of determination, denoted R2, and have been compared with the values obtained by the proposal of arterial input function sampling AIF0 subjected to many acquisition effects. Their median values were respectively of less than 0.08 in terms of mean squared error, compared with 0.15 of the samplings of the arterial input function subjected to the acquisition effects, and more than 0.85 in terms of R2, compared with less than 0.3. Thus, in the case of real acquisition, the invention made it possible to obtain a viable and correct estimation of the sampling of the arterial input function AIF sought.

For the purposes of validation of the relevance of the second arterial input function generated in step 120 by the member of healthcare personnel using a post-processing method 100 according to the invention, the latter can also include a step 150 of creating a graphic representation of said second arterial input function AIF before or after correction thereof. Such a step 150 can moreover consist of causing an output of said graphic representation by an output human-machine interface when the medical imaging analysis system implementing said method 100 includes such an output interface, like the interface 5 of the system illustrated by FIGS. 1 and 2.

The invention has been described in relation to a non-limitative example of signals originating from a cardiac acquisition sequence by the perfusion imaging device. The invention will not be limited to this single examined organ and can be used to generate pharmacokinetic parameters for any other organ of interest, such as the brain for example.

1. Method for post-processing a sampled temporal experimental signal resulting from a perfusion acquisition sequence by a medical imaging device and resulting from the passage of a tracer within an elementary volume of an organ, said method being implemented by a processing unit of a medical imaging analysis system, said method including:
a step of selecting a first arterial input function in relation to an arterial region (A0) of the organ and a set of tissue signals respectively in relation to separate tissue regions of said organ;
a step of generating a second arterial input function on the basis of said first arterial input function and of said set of selected tissue signals;
a step of creating a pharmacokinetic parameter on the basis of said second arterial input function and of said experimental signal;
wherein the step of generating a second arterial input function consists of the implementation of basic operations by said processing unit, the latter having been trained beforehand according to a process of learning the disruptive effect of the acquisition of a perfusion sequence on arterial signals and the redundancy of the information in relation to such a second arterial input function shared by a set of at least two tissue signals.

2. Method according to claim 1, including a step of correction by scaling said second arterial input function generated, before the implementation of the step of generating a pharmacokinetic parameter on the basis of said second arterial input function thus corrected and of said experimental signal.

3. Method according to claim 1, for which said medical imaging system comprises an output human-machine interface, said method including a step of creating a graphic representation of said second arterial input function and of outputting said graphic representation by means of said output human-machine interface.

4. Method according to claim 1, for which the learning process consists of deep learning based on minimization of the average value of the quadratic errors between real samples of arterial input functions which have made it possible to generate tissue signals and an estimation of these same samples performed by said learning process.

5. Method according to claim 4, for which the learning process is carried out via the Adam optimizer.

6. Computer-readable storage medium including one or more program instructions that can be executed by the processing unit of a computer, execution of which by said processing unit causes the implementation of a method according to claim 1.

7. (canceled)

8. Medical imaging analysis system including a processing unit arranged to communicate with the outside world and receive a set of samples of a temporal experimental signal, resulting from a perfusion acquisition sequence by a medical imaging device and resulting from the passage of a tracer within an elementary volume of an organ, said processing unit having been trained beforehand according to a process of learning the disruptive effect of the acquisition of a perfusion sequence on arterial signals and the redundancy of the information in relation to an arterial input function shared by a set of at least two tissue signals and including a computer-readable storage medium according to claim 6.
